(19) United States
(12) Patent Application Publication
(10) Pub. No.: US 2021/0335465 A1
(43) Pub. Date: Oct. 28, 2021

(54) SYSTEM AND METHOD FOR REMOTELY OBTAINING DONOR INFORMATION

(71) Applicant: Haemonetics Corporation, Boston, MA (US)

(72) Inventor: Melvin Tan, Edmonton (CA)

(21) Appl. No.: 17/271,473

(22) PCT Filed: Aug. 26, 2019
(86) PCT No.: PCT/US2019/048095

§ 371 Date: Feb. 25, 2021

Related U.S. Application Data
(60) Provisional application No. 62/722,933, filed on Aug. 26, 2018.

(51) Int. Cl. G16H 10/65; G16H 10/20; G06K 19/06; G06K 19/07; G16H 40/20; G06K 7/14; G06K 9/00

(52) U.S. Cl. CPC G16H 10/65; G16H 10/20; G06K 19/06028; H04L 67/12; G16H 40/20; G06K 7/1413; G06K 9/00892; G06K 19/0723

(57) ABSTRACT

A method for remotely obtaining donor information includes identifying a donor after receiving donor specific information from the donor at a remote location and retrieving, at a plasma center, a donor questionnaire based, at least in part, on the donor specific information. The method may then present at least one question from the questionnaire to the donor at the remote location and receive at least one response to question(s) from the donor. Upon receipt of the response(s), the method processes the response(s) at the plasma center and assesses a result of the processed responses to achieve a result assessment based on the at least one response. The assessment may be either a passing assessment or a non-passing assessment. The method may then encode, if the assessment is a passing assessment, the assessment and provide the donor with the encoded assessment.

### PRIORITY

This PCT patent application claims priority from U.S. Provisional Application No. 62/722,933, filed Aug. 26, 2018, entitled “Plasma Online Questionnaire,” assigned attorney docket number 130670-09401 (formerly 1611/C94), and naming Melvin Tan as inventor, the disclosure of which is incorporated herein, in its entirety by reference.

### TECHNICAL FIELD

The present invention relates to plasma donor health history information, and more particularly to the acquisition of a plasma donor's demographic and health history information from a remote location and methods of the same.

### BACKGROUND ART

Plasma donation is based on human donors in which whole blood is drawn from the donors and processed into individual blood components, such as plasma. A donor who intends to donate plasma is screened by the plasma donation center prior to being approved for a plasma donation procedure. The screening is performed on the basis of historical information and health information supplied by the donor, and this information is then assessed by a computer system and/or by staff to determine if a donor is eligible to donate plasma. All donor information required to assess a donor's eligibility to donate plasma is collected from the donor during their visit to the plasma center.

As mentioned above, donor information required for screening is collected from the donor while the donor is present at the plasma center. In some cases, the first part of the screening process is performed by the plasma donor at an electronic kiosk where questions are presented to the donor and answers are input by the donor to the electronic kiosk. The questions may in be in the form of text, pictures, audio, or video. Once all the requisite questions have been answered by the plasma donor, the computer kiosk processes the responses to assess the donor's eligibility to donate. Alternatively, the computer kiosk may delegate all or part of this assessment to a plasma center staff. Throughout the whole process, the donor must be physically present at the plasma center to partake in this screening process.

### SUMMARY OF THE EMBODIMENTS

In some embodiments of the invention, the plasma donor may perform the screening process without visiting the plasma center. Using the plasma donor's computing device such as a personal computer, laptop, cellular phone, tablet, or similar web-connected computing device, the donor can provide the answers to the screening questions from a remote location and submit the answers to the plasma center for pre-processing.

The system may begin the process by first identifying the donor so that the appropriate questions can be given to the plasma donor. The identification may consist of one or more unique information known by the donor (e.g., date of birth, social security number, first name, last name, maiden name) or information given to the donor (e.g., donor number, donor ID card, social security number). The identification may also include biometric techniques such as a fingerprint, palm scan, retinal scan, vein scan, facial recognition, body mass index, or similar physiological characterization technologies.

The questions given to the donor may be similar or dissimilar in whole or in part to the questions given to a donor while visiting the plasma center. The question responses can take on any format including: text response, multiple choice answers, multi-select answers, audio, picture, photograph, video, or any forms of electronic media or multimedia compatible to the system.

The system may enforce a time-based constraint to control the elapsed time between answering the screening questions and the plasma donor making an appearance at the plasma center. For example, the answers for the screening questions are only valid for the current day prior to the center closing or prior to midnight, whichever comes first. In another example, the answers for the screening questions are valid for a maximum of 24 hours until donation regardless of whether the donor visits the center on the current day or the next day.

The system may transmit the plasma donor's answers to the plasma center for pre-processing and assessment. The results of the plasma center's assessment of the questionnaire may be available for presentation back to the donor. Depending on the results, the donor may have one or more follow-up actions available to take.

If the system receives a passing assessment result from the plasma center, the system will encode the assessment and/or the questions and answers collected from the donor into an encoded technology such as a barcode, RFID, confirmation number, or similar passive storage technology. The encoded results are then made available to the plasma donor to be printed, to be saved on their personal computing device, or to be sent to a destination indicated by the plasma donor.

At any time, the donor may retake the questionnaire if there are any changes to the donor information. However, each questionnaire result is only valid for one visit within the time limit of the questionnaire.

When the plasma donor visits the plasma center, the encoded results can be retrieved and scanned or entered at an electronic kiosk or similar intake system provided by the plasma center to expedite retrieval of the donor's already completed questionnaire responses. At this point, the plasma donor may not be able to retake the questionnaire.

In other embodiments of the invention, the question responses are only recorded and maintained in the system and not sent to the plasma center for processing. In this method, no results will be given back to the plasma donor, but the system will still encode the questionnaire responses using the methods described above.

In accordance with some embodiments of the present invention, a method for remotely obtaining donor information includes identifying a donor after receiving donor specific information from the donor at a remote location, and retrieving, at a plasma center, a donor questionnaire based, at least in part, on the donor specific information. The method may then present at least one question from the questionnaire to the donor at the remote location, and receive at least one response to the at least one question from the donor. The method may process the at least one response at the plasma center and assess a result of the processed responses to achieve a result assessment based on the at least one response. The assessment may either be a passing assessment or a non-passing assessment. If the assessment is a passing assessment, the method may encode the assessment and provide the donor with the encoded assessment. If the assessment is a failing assessment, the method may include informing the donor of the failing assessment.

The donor specific information may be selected from the group consisting of a date of birth, a social security number, a first name, a last name, a maiden name, a donor number, and a donor ID card. Additionally or alternatively, the donor specific information may be received via biometric techniques. For example, the biometric techniques may include a fingerprint scan, palm scan, retinal scan, vein scan, facial recognition, and body mass index.

In some embodiments, the donor's responses to the questionnaire and/or the encoded assessment may be valid for a predetermined period of time. The encoded assessment may be a barcode, an RFID, and/or a confirmation number. The donor may bring the encoded assessment to the plasma center and the plasma center may receive (e.g., via an electronic kiosk at the plasma center and/or by scanning a barcode at the plasma center) the encoded assessment, and retrieve the completed donor questionnaire based on the received encoded assessment.

In accordance with further embodiments, a method for remotely obtaining donor information may include identifying a donor after receiving donor specific information (e.g., date of birth, a social security number, a first name, a last name, a maiden name, a donor number, a donor ID) from the donor at a remote location and retrieving, at a plasma center, a donor questionnaire based, at least in part, on the donor specific information. The method may then present a question from the questionnaire to the donor at the remote location and receive a response to the at least one question from the donor. Once the response is received, the method may process the response at the plasma center and assess a result of the processed response(s) to achieve a result assessment based on the at least one response. The assessment may be either a passing assessment or a non-passing assessment. If it is a passing assessment, the method may then encode the assessment and provide the donor with the encoded assessment. If the assessment is a failing assessment, the method may inform the donor of the failing assessment.

The donor specific information may be received via biometric techniques. For example, the biometric techniques may include a fingerprint scan, palm scan, retinal scan, vein scan, facial recognition, and body mass index. The donor's response to the questionnaire may be valid for a predetermined period of time. The encoded assessment may be a barcode, an RFID, and/or a confirmation number and may be valid for a predetermined period of time. For example, the method may create an expiration time for the encoded assessment

In other embodiments, the method may include (1) receiving the encoded assessment at the plasma center upon arrival of the donor at the plasma center and (2) retrieving a completed donor questionnaire based on the received encoded assessment. For example, receiving the encoded assessment may include scanning a barcode at the plasma center and/or receiving the encoded assessment via an electronic kiosk at the plasma center. Additionally or alternatively, the method may store, in a data storage device, the donor specific information, the at least one response from the donor, the result, and/or the encoded result.

In accordance with further embodiments, a system for remotely obtaining donor information includes a server, a processor and an encoder. The server may receive donor specific information from a donor at a remote location and retrieve a donor questionnaire based, at least in part, on the donor specific information. The server may also receive a response to the donor questionnaire from the donor. The processor may be in communication with the server and may process the response(s) and assess a result of the processed response(s) to achieve a result assessment. The result assessment may be either a passing assessment or a non-passing assessment. The encoder may be in communication with the server and the processor and may, if the assessment is a passing assessment, encode the response(s), the donor specific information, and/or the assessment. The server may provide the encoded information to the donor and/or provide the failing assessment to the donor.

The system may also include a data storage device that may store the donor specific information, the response(s) from the donor, the assessment, and/or the encoded result. The data storage device, the server and the encoder may be located within a plasma center. The donor specific information may include a date of birth, a social security number, a first name, a last name, a maiden name, a donor number, and/or a donor ID.

The system may also include a donor device located remotely from the server. The donor device may have an interface that allows the donor to enter donor specific information and the at least one response. The interface may include a biometric reader, and the donor specific information may be entered via biometric techniques. For example, the biometric techniques may include a fingerprint scan, palm scan, retinal scan, vein scan, facial recognition, and/or body mass index.

In further embodiments, the donor's response(s) to the questionnaire may be valid for a predetermined period of time. Additionally or alternatively, the encoded information may be a barcode, an RFID, and a confirmation number, and the encoded assessment may be valid for a predetermined period of time. The system may also include an electronic kiosk located within a plasma center. The kiosk may receive the encoded information upon arrival of the donor at the plasma center, and may be in communication with the server. The electronic kiosk may retrieve a completed donor questionnaire based on the received encoded information and may include a barcode scanner to scan the encoded information.

### BRIEF DESCRIPTION OF THE DRAWINGS

The foregoing features of embodiments will be more readily understood by reference to the following detailed description, taken with reference to the accompanying drawings, in which:

FIG. 1 schematically shows a system for remotely obtaining donor information, in accordance with various embodiments of the present invention.

FIG. 2 schematically shows a flow diagram of an online questionnaire being performed from a remote location and with connectivity to the plasma center, in accordance with some embodiments of the present invention.

FIG. 3 schematically shows a flow diagram of an online questionnaire being performed from a remote location and without connectivity to the plasma center, in accordance with some embodiments of the present invention.

### DETAILED DESCRIPTION OF SPECIFIC EMBODIMENTS

In accordance with illustrative embodiments, systems and methods may remotely obtain donor information from a donor prior to the donor arriving at a donation center and undergoing a donation procedure, for example, using a blood processing device. For example, the systems and method may identify the donor using donor specific information and present the donor with a questionnaire to complete. Based on the received donor responses the systems/methods may processes and assess the results and provide an encoded assessment if the donor receives a passing assessment.

As discussed above, prior to plasma donation, donors must be screened to ensure that the donor is a suitable donor. Additionally, the donor's physical characteristics and personal data/information may determine the volume of plasma (or whole blood or other blood components) that may be collected. For example, the FDA currently publishes a nomogram that provides guidelines concerning how much plasma (and perhaps other blood components) that may be collected from a given donor based on the weight of the donor. Furthermore, some plasma collection systems (or similar apheresis systems) determine how much plasma (or other blood component) to collect based on various patient criteria including, but not limited to, height, weight, BMI, hematocrit, total blood volume, and/or total plasma volume. In some instances, all of the required donor information may be collected upon the donor's arrival at the donation center. However, by collecting this information at the donation center, the donor is required to arrive early/in advance of the donation in order to complete the necessary questionnaire and information forms. This, in turn, increases the overall time the donor must be at the donation center and is a greater inconvenience for the donor.

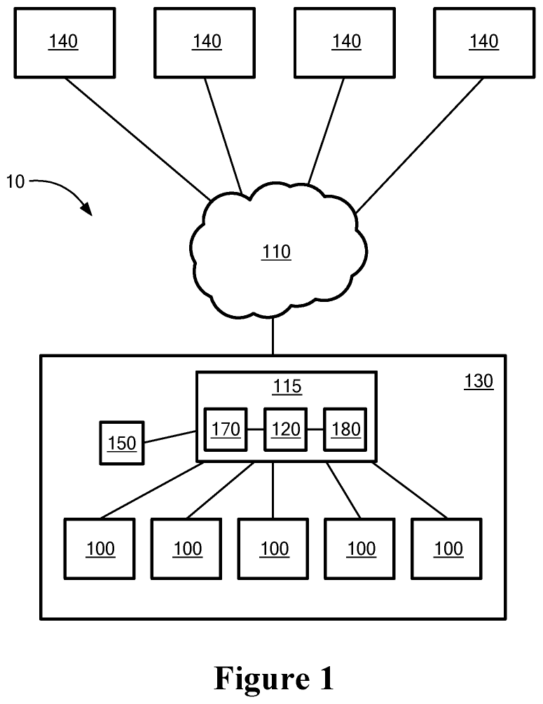

FIG. 1 shows a system 10 for remotely obtaining donor information. For example, the system 10 may include a global data communications network 110, such as the internet. The system 10 may also include a server 120 (e.g., in a plasma center computer 115) that is in communication with the global communications network 110 and, perhaps, located within a plasma center 130. In some embodiments, the server 120 supports a website that consists of a plurality of web pages that, as discussed in greater detail below, are designed to obtain the necessary information from the donor. The system 10 may also include one or more customer terminals 140 such as workstations/computers within the donor's home. Additionally or alternatively, the terminals may be the donor's laptop, cellular phone, tablet, or similar web-connected computing device.

The customer terminals 140 may be in communication with the global communications network 110 to allow the customer terminals 140 and the donor to access the server 120 and/or the website. For example, the server 120 and/or the website may be accessed and displayed by the terminals 140 (e.g., on the interface of the terminal 140) over the global communications network 110. Further, the terminals 140 may send information back to the server 120 over the global communications network 110. As discussed in greater detail below, data (e.g., data received from the donor, data relating to prior apheresis procedures, donor information, plasma center information, etc.) may be stored within a database 180 (e.g., a data storage device). Additionally, the system 10 may include an encoder 170 that encodes data received from the donor and/or the result of any analysis performed by the server 120 (or a processor within the server 120/plasma center computer 115). The encoder 170 may be located within the plasma center 130 (e.g., within the plasma center computer 115) or may be located remote from the plasma center 130.

Within the plasma center 130, the system 10 may include a number of apheresis devices like those described above (e.g., plasma collection systems 100) that may be assigned to a given donor and used to carry out the desired apheresis procedure. Each of the plasma collection systems 100 may provide data related to each of the procedures carried out to the plasma center computer 115 and server 120 (or to a server/database that is remote from the plasma center 130). In some embodiments, the server 120 may provide information back to the user/donor (e.g., to be displayed on the terminal 140 during the questionnaire process) regarding any prior donations.

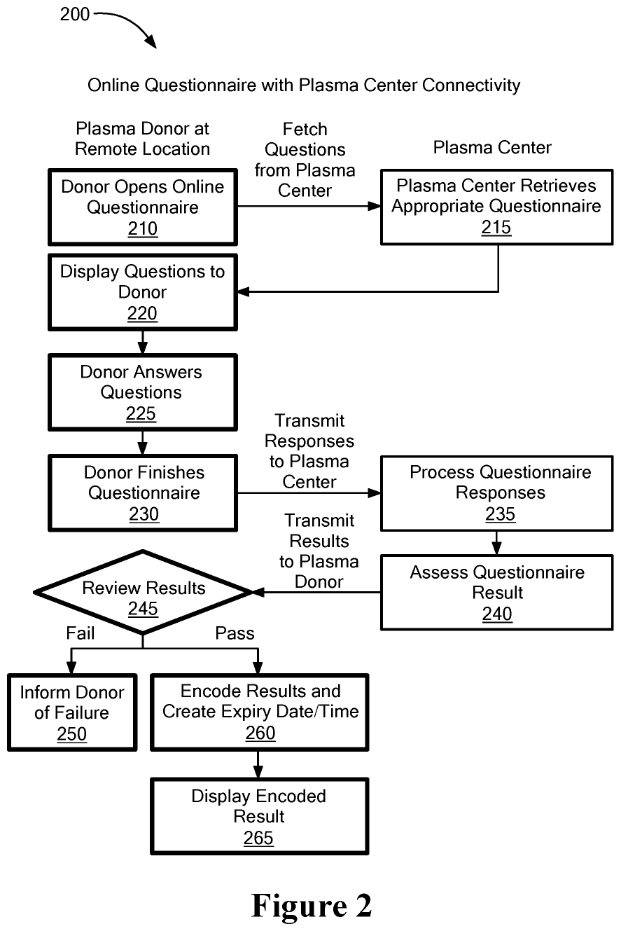

FIG. 2 schematically shows a flow diagram/method 200 of an online questionnaire being performed at a location remote from the plasma center 130, for example, to obtain the donor information. To begin, a potential donor may log onto or otherwise access an online questionnaire 210. As noted above, the donor can access the server 120 and/or the online questionnaire using any number of devices including, but not limited to the donor's personal computer, laptop, cellular phone, tablet, or similar web-connected computing device (e.g., using a terminal 140). As part of this process, the donor may provide some identification and/or donor specific information to allow the system/method 10/200 to verify the user and ensure that the appropriate questions are presented to the donor. For example, the donor may provide one or more unique pieces of information known by the donor (e.g., date of birth, social security number, first name, last name, maiden name) and/or or information given to the donor (e.g., donor number, donor ID card, social security number). Additionally or alternatively, the donor may provide the identification using biometric techniques such as a fingerprint, palm scan, retinal scan, vein scan, facial recognition, body mass index, or similar physiological characterization technologies.

The system/method 10/200 may then transmit the identification/donor specific information to the server 120 and/or the plasma center 130 (or other donation center). Upon receipt of the identification/donor specific information, the method/system 10/200 (e.g., the plasma center 130 and/or server 120) may retrieve the appropriate questionnaire based on the identification/donor specific information (Step 215) and display the questions to the donor (Step 220), for example, via the website and/or a display on the terminal 140. The questions given to the donor may be similar or different (in whole or in part) from the questions that are typically given to a donor while visiting the plasma center 130 in person. The question responses can take on any format and may depend on the type of device used by the donor to access the server 120/online questionnaire. For example, the questions and/or responses may include including: text response, multiple choice answers, multi-select answers, audio, picture, photograph, video, or any forms of electronic media or multimedia compatible with the system 10.

The donor may then answer the questions (Step 225) and complete the questionnaire (Step 230) at the terminal 140. For example, the questions may all be presented on a single page/form and the user may then enter all of the required information and answer each of the questions. Alternatively, the user may presented with only one question per page and then, once completed, the user may move onto the next question. To complete the questionnaire, the user may then select a complete and/or submit option on the website and/or the display of the terminal 140.

The system/method 10/200 may then transmit the donor's responses and/or completed questionnaire to the plasma center 130 and/or the server 120. Upon receipt, the server 120 (or the processor) will process the responses/completed questionnaire (Step 235) and will assess the data to determine the donor's eligibility to donate (Step 240). Once the server 120 has completed the assessment, the system/method 10/200 may transmit the results to the donor so that the donor can review the results (Step 245). If the assessment indicates that the donor is not eligible to donate (e.g., the assessment failed), the donor is informed of the failure result (Step 250). If the assessment indicates that the donor is eligible, the system/method 10/200 may encode the results (Step 260) and/or the answers collected from the donor using the encoder 170 and display the encoded results for viewing by the user (Step 265). The encoded result may include an encoded version of the entirety of the information provided by the donor, an encoded version of a subset of the information provided by the donor, or an encoded “code” that serves as a patient identify and allows the server 120 to later retrieve the data provided by the donor, for example, from the database 180.

The assessment and/or responses may be encoded in any number of ways including, but not limited to as a barcode, an RFID, a confirmation number, and/or similar passive storage technology. After receiving the results, the plasma donor may print the results, save the results on their personal computing device, and/or send them to a destination indicated by the plasma donor. It should be noted that, the encoding of the results in Step 260 may be carried out in any number of locations. For example, as shown in FIG. 2, the user/donor's device 140 may encode the results. Alternatively, the plasma center 130 (e.g., the server 120) may encode the results prior to sending them to the donor's device 140.

The server 120 (or the processor) may determine the donor's eligibility based on a number of criteria entered by the donor. For example, the server 120 may determine the donor's eligibility based on their age, weight, height, BMI, overall health, medical history, the date of their last donation, or a combination of any data, to name but a few. If the server 120 determines that the donor does not qualify based on one or more of the above criteria (e.g., they are not in good health, they are below a weight threshold, they donated too recently, etc.), the server 120 may provide the “failed assessment” notification to the donor.

It should be noted that the system 10 may store the responses received from the donor as well as the raw and/or encoded results. For example, the system/method 10/200 may store the information within the server 120 and/or the data storage device 180. This, in turn, allows the system/method 10/200 to easily look-up the results and/or donor information/identity, if needed, once the donor arrives at the plasma center 130, for example, to confirm the donor identity.

In some instances, it may be beneficial to place a time limit on the validity of the assessment results (particularly a passing result). To that end, the system/method 10/200 may create a time-based constraint to control the elapsed time between answering the screening questions and the plasma donor making an appearance at the plasma center 130 (e.g., the system may create an expiration date/time). For example, the answers for the screening questions may only be valid for the current day prior to the center closing or prior to midnight, whichever comes first. Alternatively, the answers for the screening questions may be valid for a maximum of 24 hours until donation regardless of whether the donor visits the center 130 on the current day or the next day. The time limit may be encoded with the assessment such that the information is scanned when the donor arrives at the donation center. Alternatively, the time limit me be stored within the data storage device 180 and then retrieved when the donor arrives at the donation center.

It should be noted that, at any time prior to arriving at the plasma donation center, the donor may retake the questionnaire if there are any changes to the donor information. This may reset the expiration deadline, but each questionnaire result may only be valid for one visit within the time limit of the questionnaire.

After the donor receives the encoded result and prior to the expiration time, the donor may bring the encoded result to a plasma center 130 at which point the encoded results can be retrieved (e.g., from the server 120 and/or database 180), scanned (via a scanner at the kiosk 150) or otherwise entered at an electronic kiosk 150 or similar intake system provided by the plasma center 130. Using the encoded result, the plasma center may retrieve the donor's completed questionnaire and responses stored within the server 120 and/or database 180. Additionally, the system/method 10/200 may also take this opportunity to confirm the donor's identity by comparing the information entered at the kiosk 150 and the information retrieved from the server 120 and/or database 180. For example, upon receipt of the encoded results, the system/method 10/200 may compare the encoded result to the information stored within the server 120 and/or database 180 (e.g., to a list of donors). If there is a match the system/method 10/200 may assign the donor to a particular apheresis device 100 and send the donor information to the assigned apheresis device 100. This, in turn, expedites the donor intake. It should be noted that in some embodiments, the donor may not be allowed to retake the questionnaire at this point.

After confirming the encoded results and/or the identity of the donor, the donor may be directed to the assigned apheresis device 100 and in some cases, the donor identity may be confirmed again at the device 100. The donor may then be hooked-up to the device 100, and the device 100 can select the appropriate apheresis program and carry out the procedure (e.g., the plasma collection procedure).

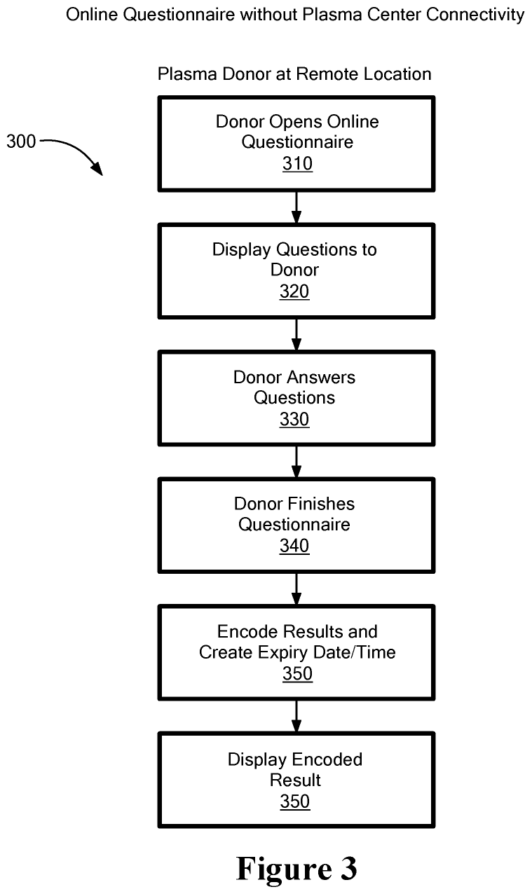

Although, the system/method 10/200 described above relates to a system/method in which the donor has connectivity with the plasma center 130 (e.g., there is an active exchange of information between the donor and the plasma center 130 throughout the process), other embodiments do not require such connectivity. For example, as shown in FIG. 3, the donor can access and complete the questionnaire without transmitting information back and forth with the plasma center 130 and without processing by the plasma center 130 (e.g., without sending and receiving information back and forth to/from the server 120, without the server 120 analyzing the data, and without the encoder 170 encoding the result). In such systems/methods 300, the user may open the online questionnaire (Step 310) and the questions may be displayed to the user (Step 320) (e.g., via software on the terminal 140, software accessed by the terminal 140, or via a standalone system that is separate from the donation center). The donor may then answer the questions (Step 330) and complete the questionnaire (Step 340). Upon completion of the questionnaire, the system/method 300 may encode the results and create the expiration date/time (Step 350) in a manner similar to that described above. The system/method 300 may then display the encoded result to the donor (Step 360). The donor may then take the encoded result to the plasma center 130 and check into the kiosk 150 as described above. It should be noted that, in this embodiment, the results of the questionnaire may or may not be provided back to the donor. Although the various embodiments above are described in relation to plasma collection and plasma centers 130, the systems/method 10/200/300 may be used for any number of donation centers and/or medical centers. For example, some embodiments may be used for whole blood donation centers, red blood cell donations or other medical centers not related to blood and/or blood component donations.

It is important to note that those skilled in the art should understand that the devices, system and method described herein may have many other physical and functional components, such as central processing units, packet processing modules, and short-term memory. Accordingly, the above discussion is in no way intended to suggest that the various embodiments described herein represent all of the elements of the system/method 10/200.

It should also be noted that the figures only schematically show each of these components. Those skilled in the art should understand that each of these components can be implemented in a variety of conventional manners, such as by using hardware, software, or a combination of hardware and software, across one or more other functional components. For example, one or more of the components may be implemented using a plurality of microprocessors executing firmware. As another example, the components may be implemented using one or more application specific integrated circuits (i.e., “ASICs”) and related software, or a combination of ASICs, discrete electronic components (e.g., transistors), and microprocessors. Accordingly, the representation of the components in a single box is for simplicity purposes only. In fact, in some embodiments, the components may be distributed across a plurality of different machines and/or locations—not necessarily within the same device.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as a pre-configured, stand-alone hardware element and/or as preprogrammed hardware elements (e.g., application specific integrated circuits, FPGAs, and digital signal processors), or other related components.

In an alternative embodiment, the disclosed apparatus, system, and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

It should be also noted that terms such as “controller,” “processor” and “server” may be used herein to describe devices that may be used in certain embodiments of the present invention and should not be construed to limit the present invention to any particular device type or system unless the context otherwise requires. Thus, a system may include, without limitation, a client, server, computer, appliance, or other type of device. Such devices typically include one or more network interfaces for communicating over a communication network and a processor (e.g., a microprocessor with memory and other peripherals and/or application-specific hardware) configured accordingly to perform device and/or system functions. Communication networks generally may include public and/or private networks; may include local-area, wide-area, metropolitan-area, storage, and/or other types of networks; and may employ communication technologies including, but in no way limited to, analog technologies, digital technologies, optical technologies, wireless technologies, networking technologies, and internetworking technologies

The various components of the control program may be implemented individually or in combination. For example, each component may be implemented or a dedicated server or a set of servers configured in a distributed manner

It should also be noted that devices may use communication protocols and messages (e.g., messages created, transmitted, received, stored, and/or processed by the system), and such messages may be conveyed by a communication network or medium. Unless the context otherwise requires, the present invention should not be construed as being limited to any particular communication message type, communication message format, or communication protocol. Thus, a communication message generally may include, without limitation, a frame, packet, datagram, user datagram, cell, or other type of communication message. Unless the context requires otherwise, references to specific communication protocols are exemplary, and it should be understood that alternative embodiments may, as appropriate, employ variations of such communication protocols (e.g., modifications or extensions of the protocol that may be made from time-to-time) or other protocols either known or developed in the future.

It should also be noted that logic flows may be described herein to demonstrate various aspects of the invention, and should not be construed to limit the present invention to any particular logic flow or logic implementation. The described logic may be partitioned into different logic blocks (e.g., programs, modules, interfaces, functions, or subroutines) without changing the overall results or otherwise departing from the true scope of the invention. Often times, logic elements may be added, modified, omitted, performed in a different order, or implemented using different logic constructs (e.g., logic gates, looping primitives, conditional logic, and other logic constructs) without changing the overall results or otherwise departing from the true scope of the invention.

The present invention may be embodied in many different forms, including, but in no way limited to, computer program logic for use with a processor (e.g., a microprocessor, microcontroller, digital signal processor, or general purpose computer), programmable logic for use with a programmable logic device (e.g., a Field Programmable Gate Array (FPGA) or other programmable logic device (PLD)), discrete components, integrated circuitry (e.g., an Application Specific Integrated Circuit (ASIC)), or any other means including any combination thereof. In some embodiments of the present invention, predominantly all of the described logic is implemented as a set of computer program instructions that is converted into a computer executable form, stored as such in a computer readable medium, and executed by a microprocessor under the control of an operating system.

Computer program logic implementing all or part of the functionality previously described herein may be embodied in various forms, including, but in no way limited to, a source code form, a computer executable form, and various intermediate forms (e.g., forms generated by an assembler, compiler, linker, or locator). Source code may include a series of computer program instructions implemented in any of various programming languages (e.g., an object code, an assembly language, or a high-level language such as FORTRAN, C, C++, JAVA, or HTML) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The computer program may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), a PC card (e.g., PCMCIA card), or other memory device. The computer program may be fixed in any form in a signal that is transmittable to a computer using any of various communication technologies, including, but in no way limited to, analog technologies, digital technologies, optical technologies, wireless technologies, networking technologies, and internetworking technologies. The computer program may be distributed in any form as a removable storage medium with accompanying printed or electronic documentation (e.g., shrink wrapped software), preloaded with a computer system (e.g., on system ROM or fixed disk), or distributed from a server or electronic bulletin board over the communication system (e.g., the Internet or World Wide Web).

Hardware logic (including programmable logic for use with a programmable logic device) implementing all or part of the functionality previously described herein may be designed using traditional manual methods, or may be designed, captured, simulated, or documented electronically using various tools, such as Computer Aided Design (CAD), a hardware description language (e.g., VHDL or AHDL), or a PLD programming language (e.g., PALASM, ABEL, or CUPL).

Programmable logic may be fixed either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), or other memory device. The programmable logic may be fixed in a signal that is transmittable to a computer using any of various communication technologies, including, but in no way limited to, analog technologies, digital technologies, optical technologies, wireless technologies (e.g., Bluetooth), networking technologies, and internetworking technologies. The programmable logic may be distributed as a removable storage medium with accompanying printed or electronic documentation (e.g., shrink wrapped software), preloaded with a computer system (e.g., on system ROM or fixed disk), or distributed from a server or electronic bulletin board over the communication system (e.g., the Internet or World Wide Web). Of course, some embodiments of the invention may be implemented as a combination of both software (e.g., a computer program product) and hardware. Still other embodiments of the invention are implemented as entirely hardware, or entirely software.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A method for remotely obtaining donor information comprising:
identifying a donor after receiving donor specific information from the donor at a remote location;
retrieving, at a plasma center, a donor questionnaire based, at least in part, on the donor specific information;
presenting at least one question from the questionnaire to the donor at the remote location;
receiving at least one response to the at least one question from the donor;
processing the at least one response at the plasma center;
assessing a result of the processed responses to achieve a result assessment based on the at least one response, the assessment being either a passing assessment or a non-passing assessment; and
encoding, if the assessment is a passing assessment, the assessment and providing the donor with the encoded assessment.

2. A method according to claim 1, wherein the donor specific information is selected from the group consisting of a date of birth, a social security number, a first name, a last name, a maiden name, a donor number, a donor ID.

3. A method according to claim 1, wherein the donor specific information is received via biometric techniques.

4. A method according to claim 3, wherein the biometric techniques include at least one selected from the group consisting of a fingerprint scan, palm scan, retinal scan, vein scan, facial recognition, and body mass index.

5. A method according to claim 1, wherein the donor's response to the questionnaire is valid for a predetermined period of time.

6. A method according to claim 1, further comprising:
if the assessment is a failing assessment, informing the donor of the failing assessment.

7. A method according to claim 1, wherein the encoded assessment is at least one selected from the group consisting of a barcode, an RFID, and a confirmation number.

8. A method according to claim 1, wherein the encoded assessment is valid for a predetermined period of time.

9. A method according to claim 1, further comprising:
receiving the encoded assessment at the plasma center upon arrival of the donor at the plasma center; and
retrieving a completed donor questionnaire based on the received encoded assessment.

10. A method according to claim 9, wherein receiving the encoded assessment includes scanning a barcode at the plasma center.

11. A method according to claim 9, wherein receiving the encoded assessment includes receiving the encoded assessment via an electronic kiosk at the plasma center.

12. A method according to claim 1, further comprising:
creating an expiration time for the encoded assessment.

13. A method according to claim 1, further comprising:
storing, in a data storage device, at least one selected from the group consisting of donor specific information, the at least one response from the donor, the result, and/or the encoded result.

14. A system for remotely obtaining donor information comprising:
a server configured to receive donor specific information from a donor at a remote location and retrieve a donor questionnaire based, at least in part, on the donor specific information, the server also configured to receive at least one response to the donor questionnaire from the donor;
a processor in communication with the server and configured to process the at least one response and assess a result of the processed at least one response to achieve a result assessment, the result assessment being either a passing assessment or a non-passing assessment; and
an encoder in communication with the server and the processor, the encoder configured, if the assessment is a passing assessment, to encode at least one selected from the group consisting of: the at least one response, the donor specific information, and the assessment, the server configured to provide the encoded information to the donor.

15. A system according to claim 14, further comprising:
a data storage device, the data storage device configured to store at least one selected from the group consisting of donor specific information, the at least one response from the donor, the assessment, and/or the encoded result.

16. A system according to claim 15, wherein the data storage device, the server and the encoder are located within a plasma center.

17. A system according to claim 14, wherein the donor specific information is selected from the group consisting of a date of birth, a social security number, a first name, a last name, a maiden name, a donor number, a donor ID.

18. A system according to claim 14, further comprising:
at least one donor device located remotely from the server, the donor device having an interface configured to allow the donor to enter donor specific information and the at least one response.

19. A system according to claim 18, wherein the interface includes a biometric reader, the donor specific information being entered via biometric techniques.

20. A system according to claim 19, wherein the biometric techniques include at least one selected from the group consisting of a fingerprint scan, palm scan, retinal scan, vein scan, facial recognition, and body mass index.

21. A system according to claim 14, wherein the donor's at least one response to the questionnaire is valid for a predetermined period of time.

22. A system according to claim 14, the server further configured to inform the donor if the assessment is a failing assessment.

23. A system according to claim 14, wherein the encoded information is at least one selected from the group consisting of a barcode, an RFID, and a confirmation number.

24. A system according to claim 14, wherein the encoded assessment is valid for a predetermined period of time.

25. A system according to claim 14, further comprising:
an electronic kiosk located within a plasma center and configured to receive the encoded information upon arrival of the donor at the plasma center, the electronic kiosk in communication with the server, the electronic kiosk configured to retrieve a completed donor questionnaire based on the received encoded information.

26. A system according to claim 25, wherein the electronic kiosk includes a barcode scanner configured to scan the encoded information.
